(19) United States
(12) Patent Application Publication
(10) Pub. No.: US 2007/0231277 A1
(43) Pub. Date: Oct. 4, 2007

(54) MULTICOMPONENT WHITENING COMPOSITIONS AND CONTAINERS

(76) Inventors: Deepak Sharma, Plainsboro, NJ (US); Janette Suh Edelstein, Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/689,013

(22) Filed: Mar. 21, 2007

Related U.S. Application Data
(60) Provisional application No. 60/788,383, filed on Mar. 31, 2006.

(51) Int. Cl. A61K 8/22

(52) U.S. Cl. 424/53; 222/144.5

(57) ABSTRACT

The present invention is directed to a container for housing and dispensing dentifrice compositions and that includes a first chamber containing therein a first dentifrice composition that includes a non-abrasive whitening agent and at least one thickener, a second chamber containing therein a second dentifrice composition that includes an abrasive polishing material, at least one thickener, a proteolytic enzyme and a rheology modifier that is not susceptible to enzymatic degradation, where the first and second dentifrice compositions are isolated one from the other until the substantially simultaneous co-extrusion from the container, and to whitening compositions containing the co-extruded first and second dentifrice compositions.

### FIELD OF THE INVENTION

This invention relates generally to dual component dentifrice whitening compositions and containers for dispensing such compositions for use on teeth.

### BACKGROUND OF THE INVENTION

Dentifrices, especially toothpaste, gels and powders containing active oxygen or hydrogen peroxide liberating ingredients, such as hydrogen peroxide, urea peroxide, percarbonates and perborates of alkali and alkaline earth metals, have been disclosed in the prior art for whitening teeth.

Non-abrasive whitening dentifrice compositions that contain peroxide compounds are known both for professional and home use. Such whitening dentifrices normally do not contain conventional abrasive or polishing agents such as silica and alumina, as such materials may activate the rapid decomposition of the peroxide compound. This is particularly undesirable where such compositions are contained within a container such a conventional tube for containing toothpaste compositions, as premature gas evolution caused by the reaction can cause swelling and/or bursting of tubes containing these products in the absence of adequate stabilization agents.

Dentifrice compositions that whiten teeth but that do not contain peroxide compounds also are known for home use and are discussed in U.S. Pat. No. 4,986,981. Such compositions utilize abrasive materials such as silica and/or alumina, in combination with a proteolytic enzyme, such as papain, an alkali-metal citrate, and citric acid, in order to aid in the prevention and removal of plaque and calculus that forms on teeth. It is stated that the use of papain in the absence of the alkali-metal citrate salt and citric acid has no affect on the calcium content of calculus.

Dual component whitening dentifrice compositions have been developed whereby the peroxide compound and the abrasive material are maintained separate from one another until just prior to application to teeth, as discussed in U.S. Pat. No. 5,766,574. Such products include the non-abrasive, peroxide-containing whitening composition in one chamber, and a composition containing an abrasive material that is not compatible with peroxide in a second chamber. However, such products do not utilize proteolytic enzymes, alkali-metal salts and citric acid in the abrasive compositions.

It would be advantageous to provide a whitening dentifrice composition that takes advantage of both the whitening affects of the peroxide-containing composition and the improved efficacy of the abrasive dentifrice compositions with respect to plaque and calculus as discussed herein above. The inventions disclosed hereunder now provide such advantageous products/systems.

### SUMMARY OF THE INVENTION

In one embodiment, the present invention is directed to a container for housing and dispensing dentifrice compositions. The container includes a first chamber having a first dentifrice composition contained therein, and a second chamber having a second dentifrice composition contained therein. The first dentifrice composition includes a non-abrasive whitening agent for whitening teeth, and a thickener, and the second dentifrice composition includes an abrasive, or polishing, material, a thickener, a proteolytic enzyme and a rheology modifier that is not susceptible to degradation by the proteolytic enzyme. The first and second dentifrice compositions remain isolated one from the other until they are substantially simultaneously co-extruded from their respective chambers of the container, at which time the first and second dentifrice compositions are combined in predetermined relative amounts to form a third composition comprising the first and second compositions for application to teeth. The invention is also directed to the dual component dental whitening compositions for home use which contain a combination of a non-abrasive whitening agent, at least one thickener, an abrasive material suitable for polishing teeth, a rheology modifier and a proteolytic enzyme.

### DESCRIPTION OF THE DRAWINGS

FIG. 1 is a rheology profile of a non-abrasive composition used in the present invention.

FIG. 2 is a rheology profile of an abrasive composition without a stabilizing rheology modifier.

FIG. 3 is a rheology profile of an abrasive composition used in the present invention containing a stabilizing rheology modifier.

FIG. 4 is a rheology profile of an abrasive composition without a stabilizing rheology modifier.

FIG. 5 is a rheology profile of an abrasive composition used in the present invention containing a stabilizing rheology modifier.

### DETAILED DESCRIPTION OF THE INVENTION

Containers and dual component dentifrice compositions as claimed utilize first and second compositions that may possess dissimilar properties, or provide differing benefits, or provide similar benefits by different mechanisms, or contain active ingredients that are incompatible with one composition or the other. The first composition is a composition containing a non-abrasive whitening agent for whitening teeth and is contained within a first chamber of a container. The second composition is an abrasive composition for the reduction and/or removal of plaque and/or calculus and is contained within a second chamber of the container and is isolated from the first composition prior to use.

The first and second compositions are then substantially simultaneously co-extruded from the container to form a third composition containing a combination of the first and second compositions. By “substantially simultaneously co-extruded”, it is meant that the first and second dentifrice compositions are extruded, i.e. dispensed, from the first and second chambers, respectively; at substantially the same time and in approximately equal volumes, or otherwise in desired predetermined relative amounts. In such systems, it is critical that the relative amount of the first and second dentifrice compositions contained in the third composition remain substantially constant upon repeated co-extrusion over time in order to provide consistent treatment of teeth from the initial application and use of the third composition use to the last application and use. Therefore, it is critical that the volumetric ratio of the first and second compositions co-extruded from the container remain substantially constant over time. By “substantially constant”, it is meant that the ratio of the first and second compositions dispensed from the container is such that the performance and the rheological properties of the third composition do not appreciably change upon repeated use of the container containing the first and second compositions.

Maintaining a substantially constant ratio of the amounts of the first and second dentifrice compositions upon co-extrusion from the container over time is important to the invention. One of the major factors in maintaining the substantially constant ratio of the first and second compositions is the rheological property of the respective compositions. As the compositions are viscoelastic, it is critical that the rheological properties of the respective first and second dentifrice compositions remain substantially constant over time. By “viscoelastic” it is meant that the compositions are both viscous and elastic. By “elasticity” it is meant that the property of a structured composition returns to an initial form or state within a specified amount of time following deformation.

Shear sensitive and thixotropic compositions, which exhibit poor or unstable elasticity, produce dual component products that are unacceptable after the composition is placed within the container due to their inability to maintain substantially constant rheological properties over time. Thus, a substantially constant ratio of the amounts of the respective components in the third composition upon co-extrusion over time may not be achieved. Poor elasticity of the composition is more of a problem in products that require careful control of the product rheology, as with compositions and containers of the present invention.

Unlike dual component systems as described in U.S. Pat. No. 5,766,574, which contain no proteolytic enzyme, the abrasive composition of the present invention comprises a proteolytic enzyme that may be capable of degrading certain thickeners.

By proteolytic, it is meant that the enzymes promote proteolysis once applied to the teeth, whereby proteins are broken down into smaller compounds. Dentifrice compositions as disclosed in U.S. Pat. No. 4,986,981, while including a proteolytic enzyme, are single component systems and, while a certain degree of degradation of thickening systems used in those compositions may occur over time, there is no issue with respect to maintaining a substantially constant ratio of the amounts of two different compositions upon substantially simultaneous co-extrusion of the compositions over time. It is only when one seeks to prepare a dual component system to be substantially simultaneously dispensed, in substantially constant ratios of the amounts of the two components over time, and where one of the components includes a thickener that may be susceptible to enzymatic degradation, that the problem associated with maintaining substantially constant rheological properties of the two components, and thus substantially constant ratio of the volumes of the two components co-extruded, is discovered. By enzymatic degradation it is meant that the enzyme breaks down and thus shortens the chains in the polymeric thickener, which results in reduced viscosity.

The present invention overcomes the problem by modifying viscoelastic properties of the second abrasive composition through the addition of a rheology modifier that is not susceptible to enzymatic degradation. The rheology modifier is present in minimum amounts effective to improve the elasticity of the compositions, but not in amounts effective to significantly impact the overall viscosity of the resultant abrasive composition. The rheological properties of the abrasive composition are thereby stabilized over time. This modification is of significance where control of the recovery of the thickening structure of the product after shear and the robustness of the rheology in the presence of proteolytic enzymes is desired.

The first dentifrice composition used according to the present invention is formulated using a vehicle containing a non-abrasive whitening agent and a thickener that is compatible with the whitening agent, optionally with pH adjusters, buffers or a zwitterionic stabilizer. The first composition typically is in the form of a gel that will have characteristics such as stand-up and viscosity similar to traditional gel dentifrice compositions. Aesthetic ingredients in this gel such as flavor and color will be chosen to be compatible with the non-abrasive whitening agent.

The first composition may include chelating agents, such as sodium ethylene diamine tetracetic acid (EDTA) or sodium acid pyrophosphate, although they are not required. The first compositions may also include additional ingredients such as humectants, surfactants, flavors and anti-caries agents, as well as other therapeutic and cosmetic agents, such as agents to treat sensitivity or to provide mineralization to the teeth.

The first compositions of the present invention include a non-abrasive dental whitening agent. Suitable dental whitening agents include, but are not limited to, peroxides, metal chlorites, metal chlorites, perborates, percarbonates, peroxyacids, and combinations thereof. Examples of suitable peroxide compounds used to prepare the first whitening component of the present invention include metal-ion-free peroxide ingredients, such as hydrogen peroxide and carbamide peroxide, and organic peroxides, such as urea peroxide, glyceryl peroxide and benzoyl peroxide, as well as metal-ion-containing peroxides, such as calcium peroxide and sodium percarbonate. A preferred peroxide compound is hydrogen peroxide. Suitable metal chlorites include calcium chlorite, barium chlorite, magnesium chlorite, lithium chlorite, sodium chlorite and potassium chlorite. Additional whitening agents include hypochlorite and chlorine dioxide and pre-cursors to these compounds. The whitening agent may be present in an amount of from about 0.01% to about 40%, by total weight of the first composition. If a peroxide compound is chosen as the whitening agent, the peroxide compound should provide an amount of hydrogen peroxide or active oxygen content equivalent to from about 0.1% to about 20%, for example from about 0.5% to about 10%, or from about 1% to about 7%, by weight of the composition. To deliver this amount of hydrogen peroxide equivalent, the peroxide compound, such as hydrogen peroxide, is generally present in an amount of from about 0.1% to about 30%, or from about 3% to about 20%, by weight of the composition.

The first compositions of the present invention also contain a thickening system containing at least one thickener. Suitable thickeners include, but are not limited to carboxypolymethylene, poloxamer, polyvinyl pyrolidone, polyvinyl alcohol, copolymers of acrylic acid such as poly(maleic acid-co-vinyl alkyl ether), methacrylic acid and mixtures thereof. Thickeners selected for thickening the first dentifrice must be stable with respect to non-abrasive whitening agents utilized in the composition, for example hydrogen peroxide. The term “carboxypolymethylene” is used to denote a broad category of polymers, particularly copolymers of acrylic acid and polyallyl sucrose. Suitable carboxypolymethylene compositions may be obtained from B. F. Goodrich Company under the tradename “CARBOPOL”. Suitable Carbopol® thickeners include Carbopol® 934, 940, 941, 956, Carbopol® ETD 2001, ETD 2020, ETD 2050, Carbopol® Ultrez 10 and mixtures thereof. Another tradename for carboxypolymethylene is CARBOMER. In its non-neutralized form, carboxypolymethylene can have a pH as low as 2.5.

The thickening system provides the primary viscosity to the composition. By primary viscosity it is meant at least about 85%, or at least about 90%, or at least about 95% of the total viscosity of the composition. The thickener(s) raise the viscosity of the first composition of the present invention to from about 200 to about 1,000,000 cps at low shear rates, e.g. less than one 1/second. Preferably, the viscosity of the compositions of the present invention is from about 100,000 to about 800,000 cps, or from about 400,000 to about 600,000 cps. The amount of thickener in the first composition may range from about 0.1% to about 50%, or from about 1% to about 15%, for example from about 1% to about 10% by weight of the composition. In most cases, polymeric thickeners naturally provide elasticity to this composition. However, while not required, a rheology modifier may be added to enhance elasticity. Such rheology modifiers can be any synthetic flexible polymers compatible with the selected whitening agent.

At least one zwitterionic stabilizer may be added to the first composition of the present invention to provide improved physical and/or chemical stability to the composition compared to a similar composition that does not contain the zwitterionic stabilizer. While not intending to be limited by the following, in aqueous solutions at pH of between about 4.5 to about 7.5, certain molecules or compounds possess zwitterionic properties and possess functional groups that can act as a proton donor as well as an acceptor in hydrogen bonding interactions. Hence these stabilizers may increase hydrogen bonding in thickened gels of the present invention, rendering them more resistant to free radical initiated degradation. The zwitterionic stabilizer may stabilize the whitening agent, e.g. hydrogen peroxide, in gels utilizing a polymeric thickener, e.g. a Carbopol® thickener, by forming a complex but favorable network of hydrogen bonds with the peroxide and/or the polymer molecules. This enhanced and complex hydrogen bonding in these gels not only arrests the degradation of hydrogen peroxide and generation of hydroxyl radicals, which in turn attack thickening polymers, but also shield the site of radical attack on the thickening polymer. The stabilizer thus aids in preventing or minimizing degradation of the whitening agent and/or the polymer thickener over time. Such degradation is associated with the decrease of pH of the dental compositions over time. A decrease in the pH of the composition indicating degradation of the polymeric thickener is associated with a reduction in viscosity of the composition. Appreciable decreases in the viscosity of the compositions then result in compositions that may not remain in close contact of the teeth for a period of time necessary to provide effective whitening, thereby resulting in decreased whitening efficacy. In addition to whitening efficacy issues, a “runny” composition may create additional aesthetic issues affecting general consumer acceptance of the product.

Zwitterionic stabilizers useful in the present invention may be in the form of a molecule, a chemical compound, or mixtures thereof. Suitable zwitterionic stabilizers include, but are not limited to, amino acids, short peptides, and their derivatives and such as glycine, di-glycine, tri-glycine, histidine, lysine, arginine, β-alanine, 4-amino butyric acid, and 6-amino hexanoic acid. Other classes of zwitterionic stabilizers include urea, betaine surfactants such as cocamidopropyl betaine, trimethylamine oxide, peptides, and combinations thereof. Selection of a suitable stabilizer may be made in view of the particular whitening agent and thickener being used, in addition to suitability for use in humans. Typically, longer chain compounds may be preferred for efficacy in that a greater distance between the respective positive and negative charges on the zwitterionic stabilizer may provide increased efficacy, although smaller compounds or molecules may be more preferable for use in humans. When used, the zwitterionic stabilizer used in the first composition of the present invention is present in an amount effective to provide improved physical and/or chemical stability to the composition. Typically, the amount of zwitterionic stabilizer may range from about 0.05% to about 5%, for example from about 0.2% to about 2%, by weight, based on total weight of the composition.

Compositions according to the present invention utilizing a zwitterionic stabilizer exhibit improved chemical and physical stability when compared to similar compositions that do not contain a zwitterionic stabilizer, even when conventional chelating agents such EDTA and/or citric acid are used. Gel compositions of the present invention typically retain at least about 75% of the formulated peroxide content when exposed to a temperature of about 40° C. over 8 weeks, or about 70% of peroxide content when exposed to 40° C. for about 12 weeks. In addition, pH of the gel compositions of the present invention typically are not reduced by more than about 25%, or not more than about 20%, when exposed to 40° C. for about 12 weeks. As noted herein above, a significant decrease in pH results in a significant reduction in viscosity of the gels of the present invention.

Humectants such as glycerin and polyethylene glycol may be used in combination with water in formulating the vehicle for the whitening component dentifrice composition of the present invention. A combination of glycerine, polyethylene glycol and water is preferred as the vehicle in which the other ingredients of the peroxide component are contained. Illustrative of polyethylene glycols useful in the present invention include polyethylene glycols known by the trademark Carbowax, which are nonionic polymers of ethylene oxide having the general formula:

HOCH₂(CH₂OCH₂)ₙCH₂OH

wherein n represents the average number of oxyethylene groups. The Carbowax® polyethylene glycols are designated by a number such as 400, 600, 800, etc., which represents the average molecular weight. Another polyethylene glycol component is available from Callahan Chemical Company under the tradename Peg-8. The average molecular weight of the polyethylene glycols used in the practice of the present invention is about 200-2,000, preferably 400-800 and most preferably about 600.

Glycerin and polyethylene glycol may be included in the first dentifrice composition of the present invention in an amount of from about 40% to about 60% by weight, and from about 5% to about 10% by weight, respectively. Water is incorporated in the aqueous whitening dentifrice compositions of the present invention at a concentration of from about 5% to about 35% by weight of the first composition, or from about 15% to about 25% by weight of the whitening composition.

In addition to glycerin and polyethylene glycol, other polyhydric solvents may further improve the chemical and physical stability of the first compositions of the present invention. Suitable polyhydric solvents include, but are not limited to, polyvinyl alcohol, polypropylene glycol, trehalose, lactose, sucrose, maltose, mannose, derivatives thereof, and mixtures thereof. When utilized, the amount of polyhydric solvent in the compositions of the present invention may range from about 0.1% to about 20%, for example from about 1% to about 10% by weight of the composition.

Chelating agents used to prepare the peroxide dentifrice component may include sodium acid pyrophosphate, disodium calcium ethylene diamine tetraacetic acid (Na₂Ca EDTA), phosphoric acid, citric acid, sodium citrate, potassium citrate, sodium pyrophosphate, potassium pyrophosphate and disodium ethylenediamine tetraacetate. The chelating agent is incorporated in the peroxide containing dentifrice component of the present invention in an amount within the range of 0.05% to about 8% by weight, or from about 0.1% to about 3% by weight of the composition.

While the first compositions of the present invention typically will be substantially non-abrasive, such that they are essentially free of or free of abrasive materials, where desired, an abrasive material that is compatible with the non-abrasive whitening agent may be incorporated in the first dentifrice component in amounts that do not materially alter the rheological properties or chemical stability of the first compositions. Examples of such abrasives include, without limitation, dicalcium phosphate dihydrate and anhydrous dicalcium phosphate or calcium pyrophosphate. Non-abrasive whitening compositions according to the present invention may be prepared by any conventional methods known for preparing such whitening compositions.

The second dentifrice composition, typically in the form of a paste, as that term is conventionally understood in the art of dentifrice compositions, comprises an abrasive material, for example, dicalcium phosphate dihydrate, insoluble sodium metaphosphate, calcium pyrophosphate, calcium carbonate, magnesium carbonate, sodium bicarbonate, hydroxyapatite, alumina, aluminosilicates, or one of a variety of silicas, including hydrated silica, dehydrated silica gels, and amorphous silica, and may contain a combination of abrasive materials where desired. Since these agents can differ in their degree of abrasiveness, both between the various types and within each type itself, the abrasiveness is carefully observed and is adjusted to a proper amount which tends to polish teeth, but not to scratch them, whether the teeth are natural teeth, or restorations. The abrasive may comprise about 10 to about 60%, or about 15% to about 35%, by weight, of the total weight of the second abrasive composition suitable for the present invention.

The second abrasive compositions also utilize a thickening system comprising at least one thickening agent to provide primary viscosity to the composition. The thickening system provides the primary viscosity to the composition with the thickener(s) raising the viscosity of the composition of the present invention to from about 200 to about 1,000,000 cps at low shear rates, e.g. less than one 1/second. Preferably, the viscosity of the compositions of the present invention is from about 100,000 to about 800,000 cps, or from about 400,000 to about 600,000 cps.

A number of agents having hydrophilic colloidal properties may be used, such as polymer hydrocolloids, polysaccharides, and mineral colloids.

Polysaccharides include pectin, natural celluloses, and natural gums such as gum tragacanth, gum karaya, and guar gum. The seaweed colloids such as various carageenans, extracts of Irish moss, and sodium alginate also may be used. Synthetic celluloses or polymer hydrocolloids, including sodium carboxymethyl cellulose and methyl cellulose, may be used. Mineral colloids such as bentonites and silicates may be used. Sodium carboxymethyl cellulose and amorphous silicates are particularly preferred to provide the bulk of viscosity to the composition. Proteolytic enzymes that may be used in the present invention may be selected from the group consisting of bromelain, ficin, trypsin, actinidin and papain.

As used herein, papain refers to the crystalline proteolytic enzyme rather than the crude dried latex. It is a preparation from commercial dried papaya latex. According to the Merck Index, the papain molecule consists of one folded polypeptide chain of 212 residues with a molecular weight of about 23,400. If papain is used, it may be incorporated in the amount of from about 0.05% to about 8%, or from about 0.1% to about 4.5%, based on the total weight of the second composition. The papain utilized in the present invention as exemplified herein has an activity of 100-800 units per milligram, as determined by the Milk Clot Assay Test of the Biddle-Sawyer Group,. (See J. Biol. Chem., Volume 121, pages 737-745,(1937)). When using a papain having a different activity, one would adjust the amount of papain to provide an overall activity essentially corresponding to compositions described herein.

In addition to the thickening system containing the thickener, the second abrasive composition utilized in the present invention includes a rheology modifier that is not susceptible to enzymatic degradation by the proteolytic enzyme and that enhances the elasticity of the composition matrix. The rheology modifier does not significantly impact the overall viscosity and is not the primary thickener of the resultant abrasive composition, although it may provide some minimal additional viscosity to the composition. The rheology modifier and the composition containing the modifier possess a high elastic modulus, an ability to rapidly retract or rebound when sheared or stretched, and a resiliency to recover original shape and/or structure once deformed. Examples of rheology modifiers that stabilize elastic rheological properties are flexible synthetic polymers, elastomers, natural binders such as xanthan gum, and hydrophobically-modified associative synthetic and natural polymers. Other natural gums that are not susceptible to enzymatic degradation also could be utilized. Flexible synthetic polymers include, but are not limited to, polyethylene, polyethylene oxides, and polyvinyl propylene.

While it may be the case that certain rheology modifiers may be generally classified or known as thickeners, as that term is understood to those skilled in the art of dentifrice compositions, when used at the levels utilized in compositions of the present invention, the rheology modifiers are not present in amounts effective to provide primary viscosity to the compositions and thus do not act as thickeners, per se. In addition, the rheology modifier will be different from thickeners used in the thickening system to provide primary viscosity in the compositions. Compositions used in the present invention comprise from about 0.1% to about 10%, or from about 0.2% to about 5%, or about 0.2% to about 3%, of the rheology modifier, based on the weight of the composition.

Typically, the present invention incorporates sodium citrate in the second composition in an amount of from about 0.5% to about 5% by weight of the total weight of the composition, or potassium citrate in an amount of from about 0.5% to about 3.3% thereof. Citric acid may be used in combination with the sodium or potassium citrate up to about 3% by weight of the total weight of the composition, although normally somewhat less is used. In certain embodiments citric acid is used at from about 0.1% to about 1%, by weight. It is used in an amount to adjust the pH of the composition to at least about 5, or between about 5.5 and about 8.

Surface-active agents may be incorporated into the second abrasive dentifrice component to provide foaming properties. The surface-active material is preferably anionic, nonionic or ampholytic in nature, and most preferably is non-ionic. Suitable examples of useful anionic surfactants are higher alkyl sulfates, such as potassium or sodium lauryl sulfate, which is preferred, higher fatty acid monoglyceride monosulfates, such as the salt of the monosulfated monoglyceride or hydrogenated coconut oil fatty acids, alkyl aryl sulfonates such as sodium dodecyl benzene sulfonate, higher fatty sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals. Examples of the last mentioned amides are N-lauroyl sarcosine, and the salts of N-lauroyl, N-myristoyl, or N-palmitoyl sarcosine. The surface-active agent is generally present at a concentration of from about 0.5 to about 5% by weight of the second abrasive dentifrice composition.

Fluorine-providing salts having anti-caries efficacy may also be incorporated in the abrasive dentifrice component of the present invention and are characterized by their ability to release fluoride ions in water. Among these materials are inorganic metal salts, for example, sodium fluoride, potassium fluoride, a tin fluoride such as stannous fluoride or stannous chlorofluoride, sodium fluorosilicate, ammonium fluorosilicate, alkyl amine fluorides and sodium monofluorophosphate. It is preferable to employ a fluoride salt to release about 10-5000 ppm of fluoride ion.

Synthetic anionic polymeric polycarboxylates optionally may be included in the abrasive dentifrice component. Polymeric polycarboxylates are well known, being often employed in the form of their free acids or preferably partially or more preferably fully neutralized water-soluble alkali metal, e.g. potassium and preferably sodium, or ammonium salts. Preferred are 1:4 to 4:1 copolymers of maleic anhydride or acid with another polymerizable ethylenically unsaturated monomer, preferably methyl vinyl ether (maleic anhydride) having a molecular weight (M.W.) of about 30,000 to 1,000,000. These copolymers are available, for example, as Gantrez® AN 139 (M.W. 500,000), AN 119 (M.W. 250,000) and preferably S-97 Pharmaceutical Grade of GAF Corporation. Other operative polymeric polycarboxylates include those disclosed in U.S. Pat. No. 3,956,480, such as the 1:1-copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Other ingredients which may be incorporated in the second abrasive dentifrice composition of the present invention include pigment, sweetener, flavor and preservative. In white dental cream formulations, the pigment may be titanium dioxide, rutile, and the proportion thereof will normally be in the range of 0.1 to about 2% by weight, preferably 0.25 to 0.75% by weight of the composition. The sweetener content will normally be that of an artificial or synthetic sweetener and the normal proportion thereof present will be in the range of 0.1 to 1% by weight, or 0.3 to 0.5% by weight of the composition. The flavor content, which is preferably of a mixed peppermint/menthol flavor, will usually be in the range of about 0.5 to about 2% by weight, or from about 0.5% to about 1.5% by weight of the composition. F.D. & C grade dyes may be used in appropriate amounts to provide desired colors.

Additional ingredients which may be incorporated in the second abrasive dentifrice component of the present invention are antibacterial agents including non-ionic antibacterial agents such as halogenated diphenyl ethers such as 2′,4,4′-trichloro-2-hydroxy-diphenyl ether (Triclosan) and phenolic compounds including phenols, and their homologs, mono- and polyalkyl and aromatic halophenols, resorcinol and its derivatives, bisphenolic compounds and halogenated salicylanilides. Examples of other antibacterial agents which may be included in the abrasive dentifrice component include thymol, eucalyptol, methylsalicylate, menthol, anethol, chlorhexidine, copper and zinc salts such as zinc citrate and sodium zinc citrate, and metronidazole, quaternary ammonium compounds such as cetylpyridinium chloride, bis-guanides such as chlorhexidine digluconate, hexetidine, octenidine and alexidine. The antibacterial agent is present in the abrasive dentifrice component in an effective antiplaque amount, typically from about 0.01 to about 5% by weight.

Anti-inflammatory agents such as ibuprofen, flurbiprofen, aspirin, indomethacin, etc., may also be included in the abrasive dentifrice component.

An anticalculus agent which is effective against calculus, such as pyrophosphate salts, hexametaphosphates, including the mono, di, tri and poly alkali metal and ammonium pyrophosphate and tripolyphosphate salts, is still another additional ingredient which may be present in the abrasive component of the present invention. Such agents are used in amounts sufficient to reduce calculus and are preferably in amounts which will release about 1% by weight. P₂O₇ ion, and preferably at least about 1.3% by weight. P₂O₇ ion. Other anti-calculus ingredients include zinc salts such as zinc chloride and zinc citrate.

Plaque buffers such as calcium lactate, calcium glycerophosphate and stronthium polyacrylates may also be included in the abrasive component. Other optional ingredients include vitamins such as vitamin A, C, E, B₆, B₁₂, K, plant extracts.

Agents useful in the treatment of dentin hypersensitivity also may be used in the present invention. Such agents include, without limitation, potassium salts such as potassium citrate, potassium chloride, amorphous calcium phosphate, potassium sulfate, potassium tartrate and potassium nitrate. Agents that will reduce hypersensitivity through dentinal occlusion may also be used such as stannous fluoride, strontium chloride, sodium and potassium oxalate as well as phosphosilicate salts.

Peroxide activators such as manganese coordination complexes, e.g. manganese gluconate, may also be incorporated in the abrasive dentifrice component of the present invention. When contacted with the peroxide ingredient of the peroxide dentifrice component, the activator compound activates the peroxide compound and accelerates the release of active oxygen to effect rapid whitening action.

The second abrasive compositions used in the present invention may be prepared by conventional methods. Containers used to house the dentifrice compositions may be of any type conventionally used in dual dentifrice component products currently known or sold.

The first and second compositions are filled into the appropriate multi-compartment container according to conventional methods utilized in the art of multi-compartment containers holding separate dentifrice compositions. Upon use, the first and second dentifrice compositions are substantially simultaneously co-extruded from the container. Upon co-extrusion of the first and second compositions of the present invention, the compositions are combined in predetermined relative amounts effective to provide a third composition suitable for polishing and whitening teeth. The co-extruded dual component composition typically will contain a predetermined relative amount of the first and second compositions, for instance a volumetric ratio of the first composition to said second composition of from about 1:3 to about 3:1, or about 1:1.5 to about 1.5:1, or about 1:1. The third composition thus synergistically combines the non-abrasive whitening agent, the abrasive polishing material and the proteolytic enzyme to provide surface and sub-surface whitening and polishing and in addition aid in the removal of and control accumulation of plaque and calculus, all at the same time. The ratio of the amounts of the first and second compositions in the third composition remains substantially constant with repeated use over time due to the stabilization of the rhelogical properties of the second abrasive composition and, in certain embodiments, physical stability of the first compositions containing a zwitterionic stabilizer. As a result, the synergistic efficacy of the third composition also remains substantially constant with repeated use, thus providing long-term consistent treatment of the teeth.

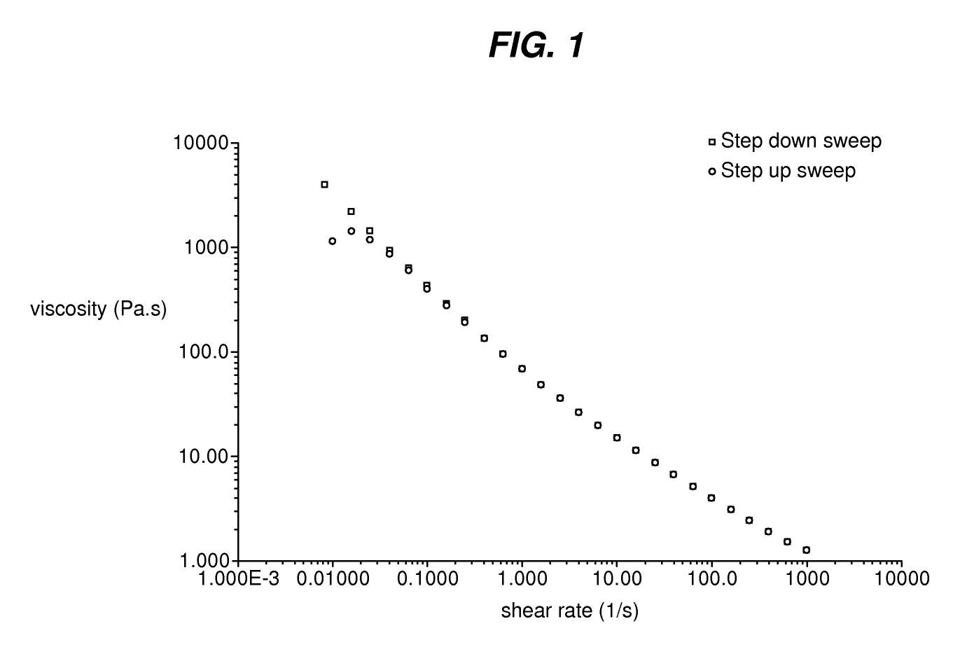

Referring to the Figures, where like symbols refer to increasing shear (step-up) and recovery profile (step-down) FIG. 1 is a rheology profile of a non-abrasive peroxide gel composition used in the present invention. As seen, the rheology profile for increasing shear is substantially the same as the recovery profile, thus exhibiting minimal hysterisis.

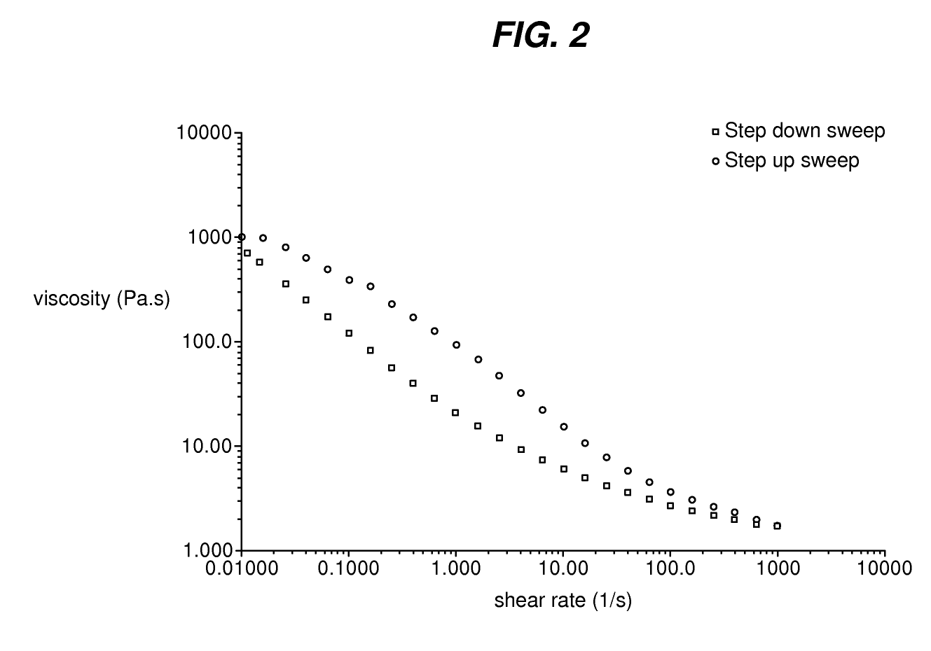

FIG. 2 is a rheology profile of an abrasive dentifrice composition comprising 0.25% papain, 0.2% carrageenan, 0.4% carboxymethylcellulose (CMC), and 1% thickening silicas, but excluding a rheology modifier that is not susceptible to enzyme degradation. All percentages are weight percent of the dentifrice composition. As seen in the profile, there is a large hysterisis loop with low elasticity and a long recovery time.

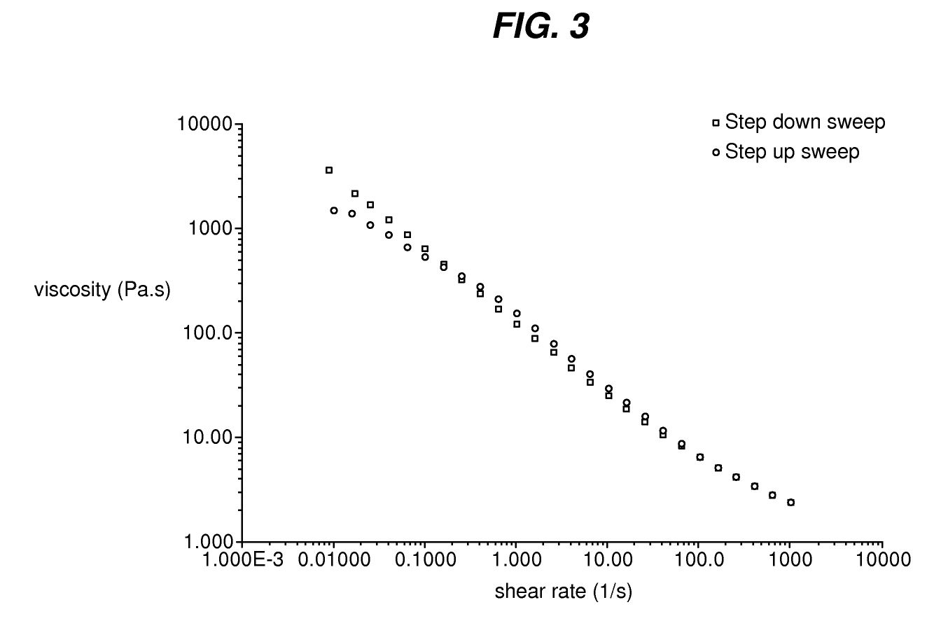

FIG. 3 is a rheology profile of an abrasive dentifrice composition used in the present invention comprising 0.25% papain, 0.5% carrageenan, 0.35% CMC, 0.2% xanthan, and 1.25% thickening silicas. All percentages are weight percent of the dentifrice composition. As with the peroxide gel, the rheology profile indicates minimal hysterisis, thus substantially matching the profile of the gel.

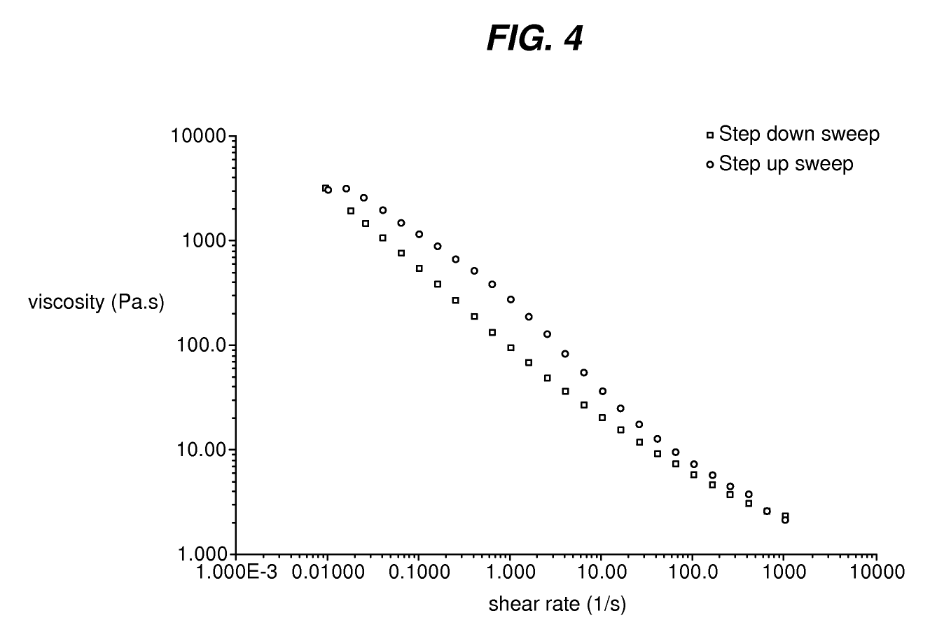

FIG. 4 is a rheology profile of an abrasive dentifrice composition comprising 0.25% papain, 0.2% carrageenan, 0.4% CMC, and 2% thickening silicas, but without a rheology modifier that is not susceptible to enzymatic degradation. All percentages are weight percent of the dentifrice composition. As seen, there is a hysterisis loop with low elasticity and a long recovery time.

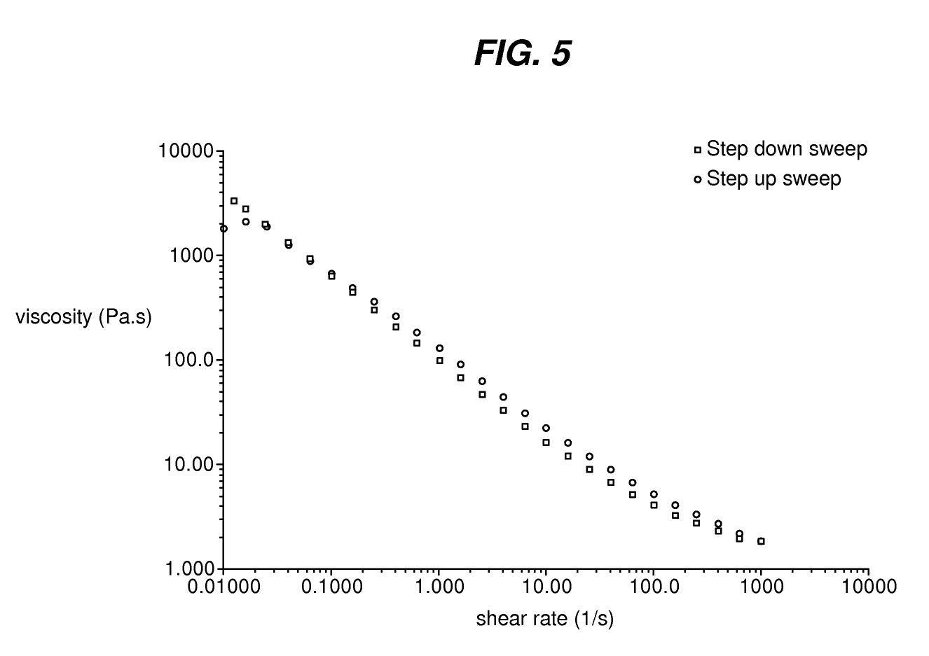

FIG. 5 is a rheology profile of an abrasive dentifrice composition used in the present invention comprising 0.25% papain, 0.5% carrageenan, 0.35% CMC, 0.2% xanthan, and 1.25% thickening silicas. All percentages are weight percent of the dentifrice composition. As with the peroxide gel, the rheology profile indicates minimal hysterisis, thus substantially matching the profile of the gel.

Examples are set forth below to further illustrate the nature of the invention and the manner of carrying it out. However, the invention should not be considered as being limited to the details thereof.

### EXAMPLE 1

Non-abrasive whitening gel compositions used in the present invention were prepared as follows.

Water-soluble components were added with mixing into a non-reactive mixing vessel containing a portion of the formulation amount of water to form an aqueous pre-mix. The residual formulation amount of water was combined with humectants in a separate vessel and mixed. Thickener was then added slowly to the separate vessel to insure proper dispersion. Once all thickeners were wetted out, vacuum was applied to minimize aeration of the composition. Heat can be used to help the thickeners hydrate. The aqueous pre-mix was added to the separate vessel containing the composition along with the zwitterionic stabilizer, where applicable. Once all ingredients were dissolved and the temperature had cooled to a temperature of about 40° C. to about 50° C., the pH was adjusted as appropriate and the flavor and surfactants were added with mixing under vacuum. The whitening agent was then added to the vessel with mixing.

### EXAMPLE 1a

45.33% purified water, 30% glycerin, 17.14% hydrogen peroxide (35% solution), 0.12% sodium saccharin, 0.16% calcium disodium EDTA, 1.8% Carbomer (CARBOPOL Ultrez 10), 3% PEG 400 hydrogenated castor oil, 0.2% L-Menthol, 1% wintergreen mint flavor, 0.8% glycine, and 0.45% potassium hydroxide (to pH 5.5).

### EXAMPLE 1b

46.13% purified water, 30% glycerin, 17.14% hydrogen peroxide (35% solution), 0.12% sodium saccharin, 0.16% calcium disodium EDTA, 1.8% Carbomer (CARBOPOL Ultrez 10), 3% PEG 400 hydrogenated castor oil, 0.2% L-Menthol, 1% wintergreen mint flavor, and 0.45% potassium hydroxide (to pH 5.5). Samples 1a and 1b were stored in capped centrifuge tubes at 40° C. Samples 1a and 1b were analyzed at various points in time for pH and hydrogen peroxide content. A significant drop in pH is associated with an undesirable decrease in viscosity of the composition. The results of the pH measurements are shown in Table 1. The results of the hydrogen peroxide content measurements are shown in Table 2.

TABLE 1

| | pH | | | | |
|---|---|---|---|---|---|
| Sample | Initial | 2 week | 4 weeks | 8 weeks | 12 weeks |
| 1a (40° C.) | 5.2 | 4.9 | 4.7 | 4.5 | 4.3 |
| 1b (40° C.) | 5.3 | 5.0 | 4.2 | 3.6 | 3.0 |

NA = not analyzed.

TABLE 2

| | Percent Hydrogen Peroxide | | | | |
|---|---|---|---|---|---|
| Sample | Initial | 1 week | 4 weeks | 6 weeks | 8 weeks |
| 1a (40° C.) | 4.0 | NA | 3.7 | NA | 3.6 |
| 1b (40° C.) | 4.0 | 4.0 | 3.7 | 0.4 | 0.1 |

NA = not analyzed.

### EXAMPLE 1c

Three gel compositions were prepared according to the procedure described above to evaluate the effect of a zwitterionic stabilizer in combination with different chelating agent such as EDTA and citric acid. Gels were formulated as noted below, with 4% by weight of hydrogen peroxide level and 0.8% by weight of glycine. EDTA or citric acid was present as indicated.

| Ingredient | Weight % |
|---|---|
| Glycerin, 99.5% USP, Glycon G100 | 50.94 |
| Water, Purified | 22.64 |
| Water, Purified | 2.5 |
| Carbopol 974P | 1.2 |
| Hydrogen Peroxide Concentrate (35%, Peralkali) | 11.43 |
| Glycine, USP | 0.8 |
| Citric Acid or EDTA | 0.5 |
| SLS Texapon | 0.3 |
| Noville Flavor AN149839 | 0.35 |
| Polyethylene Glycol 400 - NF, Carbowax Sentry | 8.0 |
| Potassium Hydroxide | 0.8 |
| Sodium Saccharin, USP, Syncal S | 0.3 |
| Sodium Fluoride, USP | 0.24 |
| | 100.00 |

The samples were stored at 40° C. and monitored for peroxide content and pH over 13 weeks. The percent formulated peroxide retention (FPR) and pH reduction data is presented in Table 3.

TABLE 3

| | Glycine + EDTA | | | Glycine + Citric acid | | | EDTA without glycine | | |
|---|---|---|---|---|---|---|---|---|---|
| Wks | % H₂O₂ | % FPR | pH | % H₂O₂ | % FPR | pH | % H₂O₂ | % FPR | pH |
| 0 | 4.00 | 100.00 | 4.96 | 3.94 | 100.00 | 4.89 | 3.9 | 100 | 4.795 |
| 1 | 3.45 | 86.35 | 4.82 | 3.81 | 96.70 | 4.86 | 3.6332 | 93.16 | 4.792 |
| 2 | 3.42 | 85.52 | 4.88 | 3.75 | 95.18 | 4.94 | 3.3306 | 85.40 | 4.744 |
| 3 | 3.35 | 83.84 | 4.99 | 3.72 | 94.42 | 4.99 | 2.9426 | 75.45 | 4.4 |
| 4 | 3.32 | 83.01 | 5.00 | 3.69 | 93.65 | 4.95 | 2.4746 | 63.45 | 4.1 |
| 5 | 3.28 | 81.89 | 4.91 | 3.61 | 91.62 | 4.99 | 2.0865 | 53.50 | 3.7 |
| 6 | 3.20 | 79.94 | 4.87 | 3.58 | 90.86 | 4.97 | 1.7328 | 44.43 | 3.4 |
| 7 | 3.25 | 81.34 | 4.72 | 3.53 | 89.59 | 4.89 | 1.4344 | 36.78 | 3.4 |
| 9 | 3.03 | 75.77 | 4.70 | 3.38 | 85.79 | 4.85 | 1.2987 | 33.30 | 3.2 |
| 10 | 2.63 | 65.74 | 4.63 | 3.37 | 85.53 | 4.52 | 1.092 | 28.00 | 3.1 |
| 12 | 2.94 | 73.54 | 4.45 | 3.20 | 81.22 | 4.66 | 0.936 | 24.00 | 3 |
| 13 | 2.89 | 72.14 | 4.40 | 3.10 | 78.68 | 4.40 | 0.702 | 18.00 | 2.9 |

As can be seen, use of the zwitterionic stabilizer inhibits reduction in pH as well as peroxide content and thus serves to provide improved stability to the first compositions. Thus, while not required for the present invention, such compositions are preferred for use in the present invention.

### EXAMPLE 1.d

24.4% purified water, 50.8% glycerin, 11.4% hydrogen peroxide (35% solution), 0.3% sodium saccharin, 2.5% Carbomer (CARBOPOL 974P), 8% PEG 8, 0.35% mint flavor, 0.3% citric acid, 0.3% sodium lauryl sulfate, 0.243% sodium fluoride, 0.8% glycine, and 0.4% potassium hydroxide (to pH 4.7)

### EXAMPLE 2

Abrasive dentifrice compositions used in the present invention were prepared as follows.

Non-aqueous humectants were added to the appropriate stainless steel vessel. Thickener(s) and rheology modifier were added and mixed until dispersed. Aqueous humectants (e.g. sorbitol) and water-soluble components (e.g. sodium fluoride, sodium saccharin, citric acid, sodium citrate, polyphosphates), colorants, and enzymes were added and mixed into the composition under vacuum. Once this composition has been adequately mixed to allow the thickening systems to hydrate and the aqueous salts to dissolve, the solid components such as silica were added in, wet and mixed under vacuum. Surfactants and flavor oils were added in the last step and mixed under vacuum.

### EXAMPLE 2a

20% deionized water, 15% glycerine, 0.5% carrageenan, 2.4% sodium citrate, 0.35% sodium carboxymethyl cellulose; 0.2% xanthan gum; 26.6% sorbitol (70% solution), 0.24% sodium fluoride, 0.25% papain, 0.4% citric acid, 0.16% methylparaben, 25% hydrated silica, 1.25% amorphous silica, 1.3% sodium lauryl sulfate, 0.5% titanium dioxide, 0.5% tetrasodium pyrophosphate, 2% doublemint flavor, 0.3% sodium saccharin.

### EXAMPLE 2b

19% deionized water, 13.8% glycerine, 0.5% carrageenan, 2.4% sodium citrate, 0.3% sodium carboxymethyl cellulose; 0.2% xanthan gum; 19% sorbitol (70% solution), 0.24% sodium fluoride, 0.25% papain, 0.4% citric acid, 0.16% methylparaben, 25% hydrated silica, 0.25% amorphous silica, 1.2% sodium lauryl sulfate, 0.5% titanium dioxide, 0.5% tetrasodium pyrophosphate, 2.5% menthol flavor, 0.3% sodium saccharin, 10% potassium nitrate, 3% tegobetaine.

Non-abrasive and abrasive compositions prepared according to Examples 1d and Example 2 were equilibrated at ambient condition and then tested for rheological profiles using a standard Brookfield viscometer (Models RVT or LVT DV-I) with the appropriate attachments and settings per recommended instrumentation manuals. Rheology shear profiles were measured using standard test methodologies for shear rate ramps and shear sweeps, preferably in logarithmic ramp mode. Thixotropic hysteresis shown in the figures are quantified by conducting two shear sweeps; the first sweep from low to high shear rate and a second shear sweep with fresh samples from high to low shear rates.

We claim:

1. A container for housing and dispensing dentifrice compositions contained therein, comprising:
a first chamber comprising a first dentifrice composition contained therein, said first dentifrice composition comprising a non-abrasive whitening agent and a at least one thickener; and
a second chamber comprising a second dentifrice composition contained therein, said second composition isolated from said first composition, said second composition comprising an abrasive material suitable for polishing teeth, at least one thickener, a proteolytic enzyme and a rheology modifier that is not susceptible to degradation by said proteolytic enzyme, said rheology modifier present in an amount effective to provide rheological stability to said second dentifrice composition.

2. The container of claim 1 wherein said non-abrasive whitening agent is selected from the group consisting of peroxides, metal chlorites, metal chlorates, perborates, percarbonates and peroxyacids.

3. The container of claim 1 wherein said non-abrasive whitening agent is selected from the group consisting of hydrogen peroxide, urea peroxide, glycerol peroxide, benzoyl peroxide, calcium peroxide, sodium peroxide, calcium chlorite, barium chlorite, magnesium chlorite, lithium chlorite, sodium chlorite, potassium chlorite, hypochlorite and chlorine dioxide.

4. The container of claim 1 wherein said non-abrasive whitening agent is selected from the group consisting of hydrogen peroxide, urea peroxide, glycerol peroxide, benzoyl peroxide, carbamide peroxide and calcium peroxide.

5. The container of claim 1 wherein said non-abrasive whitening agent comprises hydrogen peroxide.

6. The container of claim 1 wherein said first composition comprises from about 0.01% to about 40% by weight of said non-abrasive whitening agent.

7. The container of claim 5 wherein said first composition comprises from about 0.1% to about 30% by weight of said hydrogen peroxide.

8. The container of claim 1 wherein said first composition further comprises a zwitterionic stabilizer in an amount effective to provide physical and/or chemical stability to said first composition.

9. The container of claim 1 wherein said first composition further comprises from about 0.5% to about 5% by weight of a zwitterionic stabilizer.

10. The composition according to claim 9 wherein said zwitterionic stabilizer is selected from the group consisting of glysine, lysine, arginine, β-alanine, 4-amino butyric acid, 6-amino hexanoic acid, urea and trimethylamine oxide.

11. The container of claim 9 wherein said zwitterionic stabilizer comprises glycine.

12. The composition of claim 8 wherein said effective amount of zwitterionic stabilizer is effective to retain at least about 70% of formulated peroxide content in said first composition after exposure for 8 weeks at 40° C.

13. The composition of claim 8 wherein said effective amount of zwitterionic stabilizer is effective to prevent reduction of the pH of said first composition by more than about 25% after exposure for 12 weeks at 40° C.

14. The container of claim 1 wherein said rheology modifier is selected from the group consisting of synthetic polymers, elastomers, natural binders, natural gums, hydrophobically-modified associative synthetic polymers and hydrophobically-modified associative natural polymers.

15. The container of claim 1 wherein said rhelogy modifer is selected from the group consisting of polyethylene, polyethylene oxide, polyvinyl propylene and xanthan gum.

16. The container of claim 1 wherein said rheology modifier comprises xanthan gum in an amount effective to improve elasticity of said first composition.

17. The container of claim 11 wherein said rheology modifier comprises xanthan gum present in an amount of from about 0.2% to about 5% by weight of said second composition.

18. The container of claim 1 wherein said proteolytic enzyme is selected from the group consisting of bromelain, ficin, trypsin, actinidin and papain.

19. The container of claim 16 wherein said proteolytic enzyme is selected from the group consisting of bromelain, ficin, trypsin, actinidin and papain.

20. The container of claim 17 wherein said proteolytic enzyme comprises papain present in an amount of from about 1% to about 8% by weight of said second composition.

21. The container of claim 1 wherein said abrasive material is selected from the group consisting of dicalcium phosphate dihydrate, insoluble sodium metaphosphate, calcium pyrophosphate, calcium carbonate, magnesium carbonate, sodium bicarbonate, hydroxyapatite, alumina, aluminosilicates, silicates and dehydrated silica gels.

22. The container of claim 1 wherein upon substantially simultaneous co-extrusion of said isolated first and second dentifrice compositions from said first and second chambers, respectively, said first and second dentifrice compositions are combined in a predetermined relative amount to form a third dentifrice composition comprising said non-abrasive whitening agent, said abrasive material, said at least one thickener of said first and second compositions, said rheology modifier and said proteolytic enzyme is formed.

23. The container of claim 1 wherein at least one of said first and said second dentifrice composition further comprises an ingredient selected from the group consisting of a chelating agent, a flavor, a sweetener, a pigment, a therapeutic agent, a cosmetic agent, an antibacterial agent, a humectant, a surfactant, an anti-caries agent, an abrasive material that is compatible with said non-abrasive whitening agent, a pH buffer, a preservative, an anti-inflammatory agent, an anti-calculus agent, a peroxide activator, a plaque buffer and a vitamin.

24. The container of claim 1 wherein said first composition is a gel and said second composition is a paste.

25. A dentifrice composition, comprising a combination of predetermined relative amounts of:
a first dentifrice composition comprising a non-abrasive whitening agent and at least one thickener; and
a second composition comprising an abrasive material suitable for polishing teeth, at least one thickener, a proteolytic enzyme and a rheology modifier that is not susceptible to degradation by said proteolytic enzyme in an amount effective to provide rheological stability to said second dentifrice composition.

26. The composition of claim 25 wherein said non-abrasive whitening agent is selected from the group consisting of peroxides, metal chlorites, metal chlorates, perborates, percarbonates and peroxyacids, said rheology modifier is selected from the group consisting of synthetic polymers, elastomers, natural binders, natural gums, hydrophobically-modified associative synthetic polymers and hydrophobically-modified associative natural polymers, said abrasive material is selected from the group consisting of dicalcium phosphate dihydrate, insoluble sodium metaphosphate, calcium pyrophosphate, calcium carbonate, magnesium carbonate, sodium bicarbonate, hydroxyapatite, alumina, aluminosilicates, silicates and dehydrated silica gels, said proteolytic enzyme is selected from the group consisting of bromelain, ficin, trypsin, actinidin and papain.

27. The composition of claim 25 further comprising a zwitterionic stabilizer in an amount to provide physical and/or chemical stability to said first composition.

28. The composition of claim 26 further comprising a zwitterionic stabilizer selected from the group consisting of glysine, lysine, arginine, β-alamine, 4-amino butyric acid, 6-amino hexanoic acid, urea and trimethylamine oxide.

29. The composition of claim 25 comprising form about 0.01% to about 40% by weight of said non-abrasive whitening agent, based on the weight of said first composition, from about 0.05% to about 8% of said proteolytic enzyme, based on the weight of said second composition, from about 0.1% to about 10% by weight of said rheology modifier, based on the weight of said second composition, and from about 10% to about 60% by weight of said abrasive material, based on the weight of the second composition.

30. The composition of claim 29 further comprising from about 0.05% to about 5% by weight of a zwitterionic stabilizer, based on the weight of the first composition.

31. The composition of claim 30 comprising from about 0.1% to about 30% by weight hydrogen peroxide as said non-abrasive whitening agent, based on the weight of said first composition, from about 0.2% to about 2% by weight glycine as said zwitterionic stabilizer, based on the weight of said first composition, from about 0.2% to about 5% by weight xanthan gum as said rheology modifier, based on the weight of said second composition, from about 15% to about 30% silica as said abrasive material, based on the weight of said second composition, and from about 0.1% to about 4.5% by weight papain, based on the weight of said second composition.

32. The composition of claim 25 wherein said predetermined relative amount comprises a volumetric ratio of said first composition to said second composition of from about 1:3 to about 3:1.

33. The composition of claim 25 wherein said predetermined relative amount comprises a volumetric ratio of said first composition to said second composition of from about 1:1.5 to about 1.5:1.

34. The composition of claim 30 wherein said predetermined relative amount comprises a volumetric ratio of said first composition to said second composition of from about 1:3 to about 3:1.

35. The composition of claim 25 wherein said predetermined relative amount comprises a volumetric ratio of said first composition to said second composition of about 1:1.

36. The composition of claim 32 wherein said predetermined relative amount comprises a volumetric ratio of said first composition to said second composition of from about 1:1.5 to about 1.5:1.
